(19) United States
(12) Patent Application Publication
(10) Pub. No.: US 2003/0028227 A1
(43) Pub. Date: Feb. 6, 2003

(54) Locally confined photodynamic treatment for diseased tissue

(75) Inventors: Wolfgang Neuberger, Labuan (MY); Stefan Spaniol, Bonn (DE); Hans-Peter Bode, Jena (DE); Thierry Patrice, Saint Herblain (FR); Ludovic Bourre, Mauves sur Loire (FR)

Correspondence Address: CERAMOPTEC® INDUSTRIES, INC., 515 Shaker Road, East Longmeadow, MA, 01028, US

(73) Assignee: CeramOptec Industries Inc.

(21) Appl. No.: 10/224,172

(22) Filed: Aug. 20, 2002

Related U.S. Application Data
(63) Continuation of application No. 09/903,287, filed on Jul. 11, 2001, now abandoned.
Application No. 09/903,287 is a continuation-in-part of application No. 09/621,802, filed on Jul. 21, 2000, now Pat. No. 6,454,790.

(51) Int. Cl. A61N 1/00

(52) U.S. Cl. 607/88

(57) ABSTRACT

A pharmaceutical composition, method and apparatus for treatment, diagnosis or both treatment and treatment of hyperproliferative malignant and non-malignant diseases of epithelial tissues is disclosed. The invention comprises local application of the pharmaceutical composition to a predetermined area of the tissue characterized by complete and consistent coverage of the tissue, including irregularly shaped tissue. The pharmaceutical composition consists of an active component, such as a photosensitizer or a precursor thereof, and at least one carrier substance including a viscous fluid, a gel, or a fluid that becomes viscous upon contact with the tissue. The gel's viscosity allows it to adhere to the tissue for a sufficient amount of time to transfer the photosensitizer or precursor. In a preferred embodiment the pharmaceutical composition is sprayed onto the surface of the diseased tissue. Optionally, a mechanical device is used to further restrict the composition to a specific areas and can also be used to press the composition onto the tissue. In another embodiment, components of the composition are delivered to and mixed at the treatment site by a suitable delivery device prior to irradiation. The active component is then activated by a suitable wavelength of radiation.

### REFERENCE TO RELATED CASE

This application is a continuation of co-pending U.S. patent application Ser. No. 09/903,287 filed on Jul. 11, 2001 by Thierry Patrice, Wolfgang Neuberger, Hans-Peter Bode and Ludovic Bourre, inventors, entitled “Treatment for Epithelial Diseases”, which in turn was a continuation-in-part of U.S. patent application Ser. No. 09/621,802 filed on Jul. 21, 2000 by Thierry Patrice and Wolfgang Neuberger, inventors, entitled “Treatment for Barrett's Syndrome”, and incorporated by reference herein.

### BACKGROUND OF THE INVENTION

1. Field of the Invention

The present invention relates to pharmaceutical compositions, methods and apparatus for diagnosing and treating hyperproliferative pre-malignant, malignant or nonmalignant diseases of the epithelium of an organ or of the skin and tissue lying immediately below the epithelium by PhotoDynamic Therapy (PDT).

2. Invention Disclosure Statement

Hyperproliferative diseases comprise pre-cancerous or cancerous states or virally-mediated affections of body tissue, including the mucosa or interior linings of organs or the skin. There are many examples of such epithelial diseases. Barrett's esophagus is a premalignant lesion in which the normal squamous epithelium of the esophagus is replaced by a specialized columnar epithelium. Condyloma acuminata is a virally-mediated epithelial overgrowth caused by the human papilloma virus. Its papillary lesion forms are commonly seen in the genital, perineal, and anal areas. Cervical cancer or dysplasia occur at the uterine cervix. Leukoplakia is a precancerous lesion in the mouth. Actinic keratosis and basal cell carcinoma affect the skin. The diseases mentioned above occur at the epithelium of the different organs or on the skin, and thus the treatment has to reach irregular tissue structures and a certain depth of the tissue to assure a complete removal of the diseased area. In all of these cases a local treatment is desirable to reduce the side effects of the therapy and to minimize the necessary amount of the therapeutic composition. Nevertheless, locally determined but large areas of the diseased tissue should be treatable or diagnosable at one time to minimize the strain on the patient.

Photodynamic therapy (PDT) is a well-known method for the treatment of hyperproliferative diseases. A photosensitizer is applied to the patient and is activated by irradiation with (laser) light of a suitable wavelength. Upon activation of the photosensitizer, highly reactive singlet oxygen is generated which oxidizes primarily the lipids of cell membranes thereby destroying the cells and the tissue. Another well known method for photodynamic treatment of malignant and non-malignant hyperproliferative lesions is the application of an effective amount of a precursor of protoporphyrin IX (PpIX) in the biosynthetic pathway for heme so as to induce synthesis of protoporphyrin IX in the lesions, and exposing them to light having a wavelength within the photoactivating action spectrum of PpIX and thereby inducing photoactivation in the lesions for treatment or fluorescence for diagnosis. An example of such an agent is aminolevulinic acid (ALA) and derivatives thereof, which are not in themselves photosensitizers but which induce the synthesis of protoporphyrin IX (PpIX) in vivo. Protoporphyrin IX (PpIX), a naturally occurring photosensitizer, is the immediate precursor of heme in the heme biosynthetic pathway. All nucleated cells have at least a minimal capacity to synthesize PpIX, since heme is necessary for the synthesis of various essential heme-containing enzymes. However, the usual rate-limiting step in the process, the synthesis of 5-aminolevulinic acid (ALA), can be bypassed by the provision of exogenous ALA, porphobilinogen, or other precursors of PpIX. Certain tissues and organs will then accumulate such a large excess of PpIX that they become both fluorescent and photosensitive. Determination of the areas that become photosensitized is critical to reduce side effects, to reduce applied amounts of the active composition which can also reduce possible side effects, and to ensure complete treatment of the afflicted areas in all sizes and in all areas of the body.

The use of ALA for detection and treatment of malignant and non-malignant tissue abnormalities is described in U.S. Pat. Nos. 5,079,262 and 5,422,093. However, neither of these patents demonstrate a formulation that provides a local administration of the active compound to confined tissue areas, particularly in inner organs, with an even distribution of the therapeutic component.

In U.S. Pat. No. 6,034,267, esters of ALA are used in several pharmaceutical formulations for photochemotherapy of external and internal surfaces of the body. The disclosure claims to be useful for all interior and exterior epithelial surfaces. One embodiment of this invention comprises a compound consisting of the ALA esters and a carrier. Carriers given as examples include ointments and gels. The patent also provides a kit featuring separate containers for compounds, surface penetrating assisting agents and chelating agents. Although a gel is mentioned as a potential carrier in the above invention, it does not teach the use of highly viscous carrying agents to confine the active compound to specific treatment areas nor how to achieve an even distribution of the active compound on a confined tissue area. Additionally, it does not teach the spraying of a gel or thermosetting gel onto a treatment site.

U.S. Pat. Nos. 5,489,279 and 5,474,528 describe a PDT method and device for administering photosensitizers to skin by penetrating the stratum corneum. This is accomplished by the use of a hydrogel, which serves to hydrate the stratum corneum and allow radiation and photosensitizers to penetrate to the skin tissue below. Hydrogels are used because of their hydrating abilities, adhesive abilities and ability to maintain intimate contact with tissue surfaces. A skin patch-like device is described, consisting of a preformed cover, a light emitting panel, and a layer of hydrogel containing a photosensitizer. An additional embodiment describes an additional hydrogel layer containing photosensitizers that is cut to the shape of a dermal lesion (assuming such lesion is smaller than the patch) to prevent photosensitizers from entering healthy tissue.

This invention is, however, limited to external areas of the skin, and is further limited to applications where stratum corneum is present. Additionally, although the device described in this patent allows for some shaping to conform to a treatment area, it is of a fixed maximum size and shape. It is therefore unsuitable for lesions of a larger area than the patch area, in that the treatment would have to be administered numerous times to cover the entire lesion, which would be inefficient and time consuming. In the alternative, patches according to this invention would have to be available in a variety of sizes to accommodate different size lesions.

Also, the confinement properties of '279 are limited to external use, and would thus not be effective for internal epithelial layers. It would be useful to have a more flexible device and method that can be used on internal epithelial surfaces in addition to external epithelial tissue, and has the ability to allow the user to conform the patch to diseased areas of all sizes and shapes.

WO 96/06602 describes ALA skin patches and adhesives featuring a “storage stable composition” that allows storage of ALA in aqueous solutions for longer periods of time before degradation than that of previous solutions. In that formulation, ALA degrades slower, suitable for application to patient. The carrier is preferably a skin patch. This invention has no provision for restricting areas affected by ALA beside those known in the art such as skin patches. The composition does not address the concerns of the present invention, in that it provides a means for preserving ALA so as to protect it from degrading before treatment, but provides no means to confine ALA to certain areas during treatment besides a patch. It would be useful to have a means to restrict ALA and other photosensitizers that is useful for many different surfaces and does not require a patch.

The current state of the art for the treatment of Barrett's syndrome uses a Nd:YAG laser to treat the afflicted areas (Ertan et al., Am. J. Gastroenterol. 90:2201-2203[1995]). This method uses a 2.2-mm diameter beam to ablate the afflicted tissue. With such a narrow beam as compared to the size of the area being treated, the treatment procedure can be time consuming and laborious. Often the patient will have to undergo multiple procedures for a complete treatment. Also, to achieve ablation of tissue, a laser with significant power must be used. This increased power creates a larger potential for damage to the healthy tissue underneath. A 2.2 mm spot size can be relatively large for some areas, which would make it difficult to control irradiation along the edge of the diseased area. This is especially true for treatment areas that are irregularly shaped. This drawback therefore creates a potential for irradiation of surrounding healthy tissue. This method does not reveal how to treat a larger area more quickly, efficiently and more safely.

One method of treating a larger area is demonstrated in U.S. Pat. No. 6,027,499. This method uses nitrogen gas to quickly freeze the afflicted areas. Gas by its nature expands to fill the volume exposed. This expansion increases the area exposed to treatment. The freezing then kills the cells contacted. However, this method is limited by the lack of depth control in the treating of the tissue. Freezing depth is difficult to control. It would be useful to have precise control of the depth of treated tissue, so as not to damage the unaffected non-cancerous cells in the intima of the esophagus.

Gels have been used to transfer medicines at a controlled rate or to insure transfer through a tissue of the medicine. U.S. Pat. No. 4,474,752 utilizes a thermosetting gel, which gels at body temperature after injection into soft tissue. This liquid is injected into soft tissue where it gels. The gel state releases medication at a measured pace and remains in the injected area until dissolved by the body. The purpose of this invention was to create a slow release subcutaneous mechanism for medication without the discomfort of hard capsules. This invention does not reveal how to deliver medication internally to a specific site or how to restrict the medication solely to a specific treatment site.

U.S. Pat. No. 4,474,753, also utilizes a gel which solidifies on contact with the skin. This gel delivers medicine transdermally while it is attached to the skin. This is also a delivery system for medication for general release, not a site-specific medication. Also, this invention is restricted to topical application to the skin. It would be useful to provide a gel or other viscous composition to release medication both externally and internally for a specific site.

U.S. Pat. No. 4,478,822 utilizes another gel system to be injected into body cavities for dose sparing purposes. This again is a general release mechanism designed for a prolonged period of controlled drug release. The medicines released are not restricted to the area where the gel has been injected.

It would be useful to have the capability to affect a defined broad area of treatment or diagnosis with an even distribution of the active component during the application of PDT, which will quickly destroy only the afflicted area and efficiently use the photosensitive component or precursor thereof. It would be further useful to have a delivery system for medication to affect one specific area or system of a body. The present invention fills these needs.

### BRIEF SUMMARY AND OBJECTIVES OF THE INVENTION

It is an object of the present invention to provide a pharmaceutical composition, method and device for treatment, diagnosis or both treatment and diagnosis of pre-cancerous, cancerous or non-cancerous hyperproliferative states of epithelial layers and tissue in contact with the epithelium or lying immediately below using the local application of said pharmaceutical composition, characterized by sufficient localization to assure efficient uptake of the active component, and activating the active component or its metabolites with irradiation to visualize and destroy the diseased tissue.

It is a further object of the present invention to use a photosensitizer or photosensitizer precursor or a derivative thereof in the therapeutic composition and to activate the composition by light of an appropriate wavelength.

It is yet a further object of the present invention to apply the active composition in a gel form and thereby increase the area which can be treated at one time compared to other locally applied compositions, and to minimize the necessary amount of the photosensitizer while reaching a higher concentration locally than would have been achieved following a systemic administration.

It is another object of the present invention to use a fluid that becomes highly viscous or gels upon contact with the tissue or at a predetermined time after application.

It is another object of the present invention to treat diseased mucosa which is Barrett's tissue lining a patient's esophagus, esophageal dysplasia or esophageal cancer, condylomata acuminata or other types of condyloma in genital, perineal and anal areas, or other areas of the skin, leukoplakia of the oral cavity and cancer or dysplasia of the uterine cervix.

It is another object of the present invention to provide a device to deliver and evenly distribute the pharmaceutical composition, activate the active component and localize the treatment to a specific desired site for treatment, diagnosis or both treatment and diagnosis.

It is a further object of the present invention to provide a delivery device for the present invention assuring stability of any single components that are mixed directly before the application.

Briefly stated, the present invention discloses a pharmaceutical composition, method and apparatus for diagnosis, treatment or both diagnosis and treatment of hyperproliferative malignant and non-malignant diseases of epithelial tissues and tissue in contact with the epithelium or lying immediately below. The invention comprises local application of the pharmaceutical composition to a predetermined area of the tissue characterized by complete and consistent coverage of the tissue, including irregularly shaped tissue. The pharmaceutical composition consists of an active component, such as a photosensitizer or a precursor thereof, and at least one carrier substance including a viscous fluid, a gel, or a fluid that becomes viscous upon contact with the tissue. The gel's viscosity allows it to localize and promote transfer of the active ingredient to the diseased tissue, and avoid contact with healthy tissue, for a sufficient amount of time to transfer the photosensitizer or precursor. In a preferred embodiment the pharmaceutical composition is sprayed onto the surface of the diseased tissue. Optionally, a mechanical device is used to further restrict the composition to a specific area and can also be used to press the composition onto the tissue. In another embodiment, components of the composition are delivered to and mixed at the treatment site by a suitable delivery device prior to irradiation. The active component is then activated by a suitable wavelength of radiation.

The above, and other, objects, features and advantages of the present invention will become apparent from the following description read in conjunction with the accompanying drawing.

### BRIEF DESCRIPTION OF FIGURES

FIG. 1—A side view of a skin patch.

FIG. 2—a drawing of a preferred embodiment of a delivery device.

### DETAILED DESCRIPTION OF THE PREFERRED EMBODIMENT

It is an object of the present invention to locally apply a pharmaceutical composition to a selected area of a diseased epithelium, to confine said active composition to said areas, to allow time for sufficient uptake and to activate the composition to detect and/or destroy the diseased tissue.

In a preferred embodiment, aminolevulinic acid (ALA) or derivatives thereof are used as active components from which clinically useful amounts of protoporphyrin IX (PpIX) can be synthesized within the tissues, and the provision of ALA is only beneficial if the tissue affected is at a site that can be reached by photoactivating light. ALA is water soluble and can be administered orally, topically or by injection. There are a number of advantages to using ALA. First, the biosynthesized PpIX has a much shorter half-life in normal tissues than does Hematoporphyrin IX (HpIX), Hematoporphyrin derivative (HpD) or Photofrin II. This greatly reduces the danger of accidental photo toxic skin reactions in the days following treatment and/or diagnosis. Second, topical application of ALA to certain types of lesions can induce PpIX exclusively within those lesions. This improves the specificity of the treatment and/or diagnosis, reduces the danger of accidental photo toxic tissue reactions to a very low level, and greatly reduces the amount of both ALA and PpIX to which the entire body would be exposed if an equally effective dose of ALA were to be given systemically. Both ALA and PpIX are normal products of metabolism, and are handled quite readily by the biochemical machinery of the body. However, since very large doses of ALA (like large doses of HpIX or HpD) are associated with a transient decrease in motor nerve conduction velocity, it is desirable to reduce the dose of ALA to the minimum that is still effective. Topical application requires much less ALA than systemic administration. Third, PpIX quickly becomes inactiveable by the photoactivating light after treatment. Following exposure of tissues containing PpIX to a therapeutic dose of photoactivating light, there is a substantial decrease in photosensitization of the tissues within the treatment volume. Consequently, if PpIX is induced by the topical application of ALA to specific lesions, the patient can be exposed to sunlight immediately after treatment without danger of serious photo toxicity. However, to also reach deeper parts of the afflicted tissues, it might be useful to apply photosensitizers that are activated by longer wavelengths than PpIX is, because of the deeper penetration of longer wavelengths into the tissue. Therefore, in another preferred embodiment the use of “second generation photosensitizers” including porphyrins, chlorins, pheophorbides, bacteriopheophorbides and derivatives thereof are used for treatment of the diseased epithelial areas and tissue lying below.

Local treatment of specific epithelial areas, preferably large areas, is achieved by application of the active component, such as a photosensitizer or its precursor, in a pharmaceutical composition with a carrier substance including a highly viscous fluid. Such highly viscous fluid encompasses a gel, a fluid which gels upon contact with the tissue such as a thermosetting gel, and a fluid which gels after a set period of time. It has been shown that uptake of ALA into tissue from a thermosetting gel is significantly enhanced compared to application of watery ALA solutions (see example 1). The viscosity of the gel allows it to localize on the desired tissue for a sufficient amount of time to efficiently transfer the photosensitizer or the photosensitizer precursor, allows precise application to large predetermined areas, and also limits further distribution of the active component.

The mechanism for the delivery of the photosensitizers or precursors involves a few basic functions. First, the fluid or gel must be delivered to the treatment site. In the case of easily accessible treatment sites the gel can be applied directly. Where treatment is of the interior lining of inner organs, the gel can be applied through a catheter as a premixed gel or through a double lumen catheter and mixed at the treatment site. In place of a straight gel, a liquid that gels after contact with the afflicted surface can serve the same purpose in delivering the photosensitizer to the treatment site. In an alternative embodiment, separate components can be combined at the treatment site to form a highly viscous fluid. It is advantageous to spray this liquid onto irregularly structured tissue to achieve an even distribution of the active component. Moreover, the sprayed fluid generally gels in a shorter time resulting in better localization and more efficient delivery of the active component. This is a distinct advantage over conventional applications of liquid solutions of photosensitizers through spraying, which result in a loss of material and in unwanted side effects. The spraying of a liquid photosensitizer to the treatment area is an inefficient method of saturating the tissue in that the liquid will drip off the epithelium into organs or on areas of the body that are not to be treated. The liquid nature of typical photosensitizers also creates a problem in controlling the treatment area. Dripping may cause areas of the same organ or other nearby organs to receive unwanted treatment. In contrast, the gel as used in the present invention is prevented from moving beyond the treatment site by its viscosity.

Further embodiments are contemplated to control the point in time in which the composition increases in viscosity. In certain areas, it may be advantageous to delay gelling of the liquid. In one embodiment, components of the composition are kept in separate compartments in, for example, a double lumen catheter. A user can thus control when the composition increases in viscosity by controlling when the components are mixed. In a further embodiment, thermosetting gel is sprayed on a treatment site. A means for blowing cool air or other gas onto the site is also provided. The cool air or gas prevents the thermosetting gel from increasing in temperature upon contact with the tissue, and thus increasing in viscosity. By regulating the application and/or temperature of the air or gas, the user can control the temperature of the thermosetting gel and thus control the viscosity.

After the photosensitizer or precursor is delivered via the above-described carrier and allowed to penetrate the diseased epithelial layer, electromagnetic radiation of a proper wavelength is applied to the layer to activate the photosensitizer. A variety of sources may be utilized to produce such radiation, including laser sources, chemiluminescent sources, and radio frequency generators. In a preferred embodiment, especially for interior epithelial layers, radiation is delivered through one or more optical fibers, preferably via a catheter or endoscopic device. Additionally, in another embodiment, electrical and magnetic fields are utilized to improve the transfer of active molecules through the electrical field.

Excess or spent gel can be removed through a vacuum or spent gel may also be allowed to be excreted. The procedure can be viewed through fiber optic bundles passed through a catheter.

The following descriptions of devices are incorporated into the present invention and demonstrate the flexibility of the present invention. A variety of devices may be used to deliver the pharmaceutical compound to many different areas of the body. The following embodiments demonstrate how the present invention can be used both for internal and external epithelial diseases.

In one embodiment for application of the present invention to external epithelia, the pharmaceutical composition is applied to affected areas of the skin with the aid of an improved skin patch. This assures a prolonged and protected application of the pharmaceutical composition on a confined area. Although skin patches are known in the art for delivery and restriction, none address the issues presented in the embodiments below. The skin patch used in conjunction with the present invention is primarily a means for delivering and separating components of a pharmaceutical compound prior to introducing the active component to a treatment area, rather than a means for localizing the active component during treatment as in the prior art. A skin patch as presented in the present invention performs a number of functions:

1. It keeps components separate to prevent degradation of the active compound before treatment or diagnosis;

2. localizes components in an area during mixing; and

3. protects the compound from various environmental conditions during uptake or potentially during treatment or diagnosis.

A skin patch used in conjunction with the present invention is distinguishable from the prior art in that the patch itself is not the primary means for localization of the active component. A highly viscous carrier performs this function in the present invention. Additionally, a patch according to the present invention can be easily modified to conform to the shape and size of a treatment area, and is not restricted in size as in the prior art.

In a preferred embodiment, the skin patch is designed such that different layers provide light protection or light transparency as necessary during treatment and/or diagnosis. For administration of the pharmaceutical composition, light protection is desirable to maintain the stability of the active component. For activation of the active component or its metabolites in the skin after the uptake period the light protecting layer is removed, and a transparent layer allows irradiation of the treatment area. Additionally, the present skin patch need not be retained after mixing, because the carrier's viscosity serves to localize the active component during uptake. The patch's primary purpose is to aid in accurately applying the compound to a diseased dermal area and to maintain components separately prior to mixing.

In another preferred embodiment the skin patch is prepared so that the components of the pharmaceutical composition are separated on the patch and become mixed upon removal of a protective layer of the skin patch or by other known means. Separation of the components in some cases is essential to maintain the stability of some of the components and to protect them from degradation during delivery, transport and storage. The separation is provided by a rupturable chamber containing one component which is opened e.g. by pressure and mixed with the other component. A separate layer can then be removed to allow the active component of the composition to penetrate the tissue.

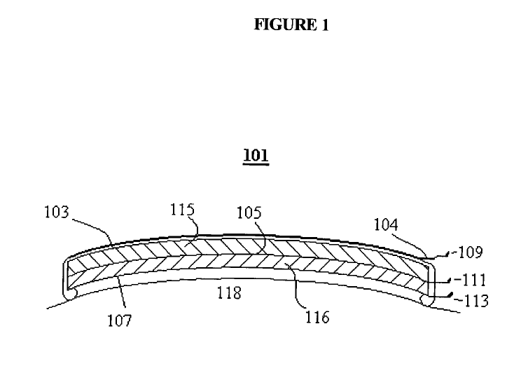

In a preferred embodiment, for example, a skin patch consists of three layers. An example of a preferred embodiment of a skin patch in accordance with the present invention is illustrated in FIG. 1. Skin patch 101 consists of three layers. Transparent outer layer 103 protects the components of the pharmaceutical composition from the environment prior to treatment and/or diagnosis, and film 104 protects the components from light. Separation layer 105 resides between the components to keep them separated, and inner layer 107 separates the components from the skin surface. Pull-tabs 109, 111 and 113 are connected to film 104, layer 105 and layer 107, respectively. In the current example, the components consist of thermosetting gel 115 and an aqueous solution of ALA 116. Other components can also be used, for example, dry ALA and a solution to dissolve the ALA upon removal of separation layer 111. Additionally, more than one separation layer is envisioned. For example, another embodiment would consist of a patch with separate compartments for dry ALA, an aqueous solution, and a gel.

In use, patch 101 is placed over diseased skin 118. Separation layer 105 is removed with pull-tab 111 to allow ALA solution 115 and thermosetting gel 116 to mix. After sufficient time has passed for the components to mix, inner layer 107 is removed with pull tab 113 to allow the composition to contact the skin and for the active component to penetrate the tissue. After sufficient time is allowed for the active component to penetrate tissue, Film 104 is removed with pull-tab 109 to allow treatment radiation to penetrate through outer layer 103 to activate the active component. In yet another embodiment, patch 101 is removed entirely, and the active component is held in place solely by the viscous gel. A protective covering to shield the area from radiation prior to treatment and/or diagnosis would still be used.

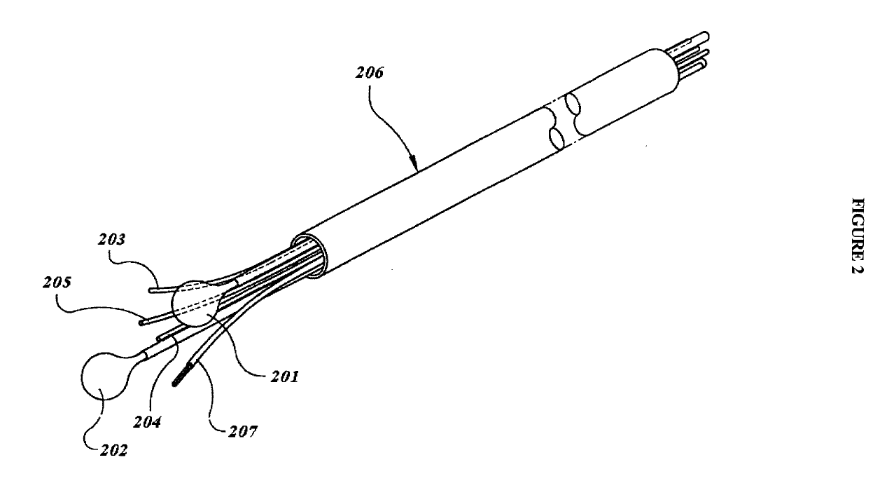

Another preferred embodiment of the invention includes a delivery device for the pharmaceutical composition separating the different components of the pharmaceutical composition to prevent degradation and to maintain stability of the components. It has been shown that ALA as well as other photosensitizers have a limited stability in solutions. This device, which can take the form of a specialized skin patch as described above for external use or a multi-lumen catheter for internal use (see FIG. 2), can also serve to guide and deliver radiation and other components such as a vacuum line or a device such as a diaphragm to further restrict application of the photosensitizer or precursor.

In such embodiments, the delivery device of the present invention provides different connected chambers for the components of the pharmaceutical composition, whereby the connections are closed during transport and storage of the product, and are opened directly before use by a simple mechanism to allow the mixing of the components. Preferably the chambers are evacuated to assure that no air bubbles affect the consistency of the gel during mixing the components. In one preferred embodiment, the delivery device is a two chamber device with solid ALA in one chamber and the prepared gel in water in the other chamber. For enhanced dissolution of the solid ALA it may be advantageous to provide an additional chamber with water to first dissolve the ALA before opening the connection to the third chamber with the prepared gel. Also, the separation of all the above components is conceivable. The delivery device can be fabricated from flexible synthetic material that can preferably be evacuated. Also the use of a syringe with different chambers to ascertain defined mixing by moving a piston is conceivable as an embodiment within this class.

An example of such a delivery device is illustrated in FIG. 2. This device is more fully described in U.S. patent application Ser. No. 09/621,802 by Patrice et al, of which the present application is a continuation-in-part.

The current example of FIG. 2 is specifically suitable for treating and/or diagnosing diseased esophageal mucus, although the general characteristics described are applicable for other interior epithelial layers. Flexible catheter 206 comprises numerous separate lumens for delivery of treatment radiation, various pharmaceutical composition components and other therapeutic substances or compounds. Radiation lumen 203 contains one or more optical fibers for delivery of treatment radiation, and image lumen 207, containing an optical fiber image bundle, may also be included. The pharmaceutical compound featuring a highly viscous gel is delivered via gel delivery lumen 205. In another preferred embodiment, lumen 205 consists of separate chambers to keep the gel and the photosensitive compound or solution separate. In this embodiment, the components are mixed within lumen 205 prior to application of the pharmaceutical compound. A spray nozzle is preferably attached to the distal end of lumen 205 in order to evenly spray the compound on the treatment site. Optional components include vacuum tube 204 for removal of excess or spent gel, and balloons 201 and 202 for restricting the gel to a specified area in the esophagus.

The present invention is further illustrated by the following examples, but is not limited thereby.

### EXAMPLE 1

### Improved Uptake of ALA into Murine Stomach Mucosa from Thermosetting Gel Preparations

ALA was incorporated into Noveon® and Pluronic® F-127 gels at concentrations varying from 2.5 to 40 mg/mL to obtain ALA-Noveon® and ALA-Pluronic®. An abdominal incision was performed on the rodent, followed by pylorus ligature and washing of the stomach mucosa with an aqueous solution of NaCl (0.9%, 37° C.). One milliliter of gel was then instilled directly through an 18-gauge needle into the stomach lumen. After a period of 30 min to 4 hours of instillation, the gel was removed and the stomach was washed with NaCl aqueous solution. The fluorescence of Protoporphyrin IX synthesized by cells of the stomach mucosa was detected by laser fiber spectrofluorimetry. Measurements were performed through an optic fiber placed in direct contact with the stomach wall. A control of the optical properties of the different gels had previously been performed using the same excitation wavelength. Results were expressed in counts per second. All experiments were performed, and data was collected from, live anesthetized animals. At 2.5 mg/mL concentration of ALA, a better uptake has been measured at any time for ALA-Pluronic (gel compared to ALA-Noveon® or ALA only. For example, after 4 hours of contact between the gel and gastric mucosa, the PpIX signal after incubation with ALA-Pluronic® was 15% higher than compared to ALA-Noveon®, and 65% than for the aqueous solution of ALA.

### EXAMPLE 2

### Diagnosis of Skin Diseases

The pharmaceutical composition was prepared with Lutrol and 5-aminolevulinic acid (ALA) in water. The thermosetting gel was prepared with 17.9% Lutrol in water by stirring under cooling. It was shown that the viscosity of the gel increased with increased concentrations of ALA. Several concentrations in the range of 2 to 40% (w/w) ALA were tested by detecting the fluorescence of protoporphyrin IX on skin. A concentration of 2% ALA only showed very low fluorescence on healthy skin areas compared to diseased skin areas. In general, the fluorescence was very low for this concentration. Therefore, for clinical studies a concentration of ALA of 10% was used, and the duration of incubation was reduced compared to the therapeutic approach (see example 3). The fluid was sprayed onto the areas of the skin to be examined to achieve an even distribution of the active component on a broad area, and the thermosetting gel became highly viscous upon contact with the warm skin and assured maintenance of the distribution. The active component was allowed to be absorbed for 1 hour under light protection to reduce its photodegradation, the gel was removed and a second incubation period followed before detection.

### EXAMPLE 3

### Treatment of Basal Cell Carcinoma and Actinic Keratosis of the Skin

The pharmaceutical composition was prepared as described in Example 2. A concentration of 2% ALA only showed very low fluorescence on healthy skin areas compared to diseased skin areas. This is desirable for therapeutic use, however with the low content of ALA one cannot be sure that the active composition penetrates deep enough into the tissue. Therefore and because of the increased viscosity of the gel, higher concentrations of ALA are preferred for therapeutic use. With a concentration of 10% ALA, significantly enhanced fluorescence could be detected which was not significantly increased by concentrations of ALA of 20 or 40%. For the clinical studies, the Lutrol gel with 10% ALA, dissolved directly before use, was applied for 2 hours under light protection. Then the gel was removed and the incubation continued for an additional 2 hours. Lastly, the treatment areas were irradiated at 633 nm with an energy density of 100-150 J/cm² and a power density of 0.05-0.35 W/cm². Multiple basal cell carcinoma lesions were treated successfully, showing superficial necrosis and very good cosmetic results.

### EXAMPLE 4

### Treatment of Diseased Tissue at the Uterine Cervix

Treatments of diseased cervical tissue can be performed in a similar fashion as the above examples. ALA is incorporated into gels that are then introduced into the diseased portion of the cervix. In addition to the gel's high viscosity, additional measures are employed to enhance the tendency of the gel to remain localized. In a preferred embodiment, a rubber cup similar to a contraceptive cervical diaphragm is placed in the cervix to further restrict the composition. In another preferred embodiment, a moist dressing especially designed for mucosal surfaces is utilized. After a sufficient uptake period, the additional localization measure and the gel is removed.

Having described preferred embodiments of the invention, with reference to the accompanying drawing, it is to be understood that the invention is not limited to the precise embodiments, and that various changes and modifications may be effected therein by skilled in the art without departing from the scope or spirit of the invention as defined in the appended claims.

What is claimed is:

1. A pharmaceutical composition for diagnosis and treatment of diseased tissue, including diseased epithelium and affected tissue lying immediately below said epithelium, comprising:
an active component; and
at least one carrier substance;
wherein said carrier substance has sufficient viscosity to remained confined to a specific area.

2. A pharmaceutical composition according to claim 1, wherein said active component is selected from a group consisting of a photosensitizer, photosensitizer precursor, aminolevulinic acid (ALA) and derivatives of ALA.

3. A pharmaceutical composition according to claim 1, wherein said carrier substance is a highly viscous fluid.

4. A pharmaceutical composition according to claim 1, wherein said carrier substance is a gel.

5. A pharmaceutical composition according to claim 1, wherein said carrier substance is a fluid that becomes highly viscous after said substance is applied to said tissue.

6. A pharmaceutical composition according to claim 4, wherein said carrier substance is a thermosetting gel.

7. A method for treatment of diseased tissue including diseased epithelium and affected tissue lying immediately below said epithelium comprising the steps of:
locally applying a pharmaceutical composition as defined in claim 1 to a selected area of said diseased epithelium;
confining said pharmaceutical composition to said area;
allowing time for sufficient uptake of an active component into said diseased tissue; and
activating said component and metabolites thereof, thereby destroying said diseased epithelium and affected tissue lying immediately below.

8. A method for treatment of diseased epithelium and affected tissue lying immediately below according to claim 7, comprising the further step of flushing said carrier substance away from said epithelium between said uptake step and said activating step.

9. A method for treatment of diseased epithelium and affected tissue lying immediately below according to claim 7, comprising the further step of mixing components of said pharmaceutical composition just prior to application step.

10. A method for treatment of diseased epithelium and affected tissue lying immediately below according to claim 7, where said activating step consists of irradiation of said component with an appropriate wavelength to activate said component.

11. A method for treatment of diseased epithelium and affected tissue lying immediately below according to claim 7, wherein said diseased tissue is mucosa lining a patients' esophagus affected by Barrett's Syndrome.

12. A method for treatment of diseased epithelium and affected tissue lying immediately below according to claim 7, wherein said disease is selected from the group consisting of condylomata acuminata, condyloma of the genitals, cervix, perineum, anal areas, and skin.

13. A method for treatment of diseased epithelium and affected tissue lying immediately below according to claim 7, wherein said disease is selected from a group consisting of leukoplakia of the oral cavity, cancer of a uterine cervix, dysplasia of a uterine cervix, and basal cell carcinoma of the skin.

14. A method for treatment of diseased epithelium and affected tissue lying immediately below according to claim 7, wherein said application step consists of spraying said composition to achieve an effective and even coverage of said diseased epithelium.

15. A method for diagnosis of diseased tissue including diseased epithelium and affected tissue lying immediately below said epithelium comprising the steps of:
locally applying a pharmaceutical composition as described in claim 1 to a selected area of said diseased epithelium;
confining said pharmaceutical composition to said area;
allowing time for sufficient uptake of said active component into said diseased tissue; and
activating said component and metabolites thereof, thereby detecting said diseased epithelium and affected tissue lying immediately below.

16. A method for diagnosis of diseased tissue including diseased epithelium and affected tissue lying immediately below according to claim 15, comprising the further step of flushing said carrier substance away from said epithelium between said allowing step and said activating step.

17. A method for diagnosis of diseased tissue including diseased epithelium and affected tissue lying immediately below according to claim 15, comprising the further step of mixing components of said pharmaceutical composition prior to application step.

18. A method for diagnosis of diseased epithelium and affected tissue lying immediately below according to claim 15, where said activating step consists of irradiation of said component with an appropriate wavelength to activate said component.

19. A method for diagnosis of diseased tissue including diseased epithelium and affected tissue lying immediately below according to claim 15, wherein said diseased tissue is mucosa lining a patients' esophagus affected by Barrett's Syndrome.

20. A method for diagnosis of diseased tissue including diseased epithelium and affected tissue lying immediately below according to claim 15, wherein said disease is selected from the group consisting of condylomata acuminata, condyloma of the genitals, cervix, perineum, anal areas, and skin.

21. A method for diagnosis of diseased tissue including diseased epithelium and affected tissue lying immediately below according to claim 15, wherein said disease is selected from a group consisting of leukoplakia of the oral cavity, cancer of a uterine cervix, dysplasia of a uterine cervix, and basal cell carcinoma of the skin.

22. A method for diagnosis of diseased tissue including diseased epithelium and affected tissue lying immediately below according to claim 15, wherein said application step consists of spraying said composition to achieve an effective and even coverage of said diseased epithelium.

23. A device for diagnosis and treatment of diseased epithelium and tissue lying immediately below for use in the method of claim 1, comprising:
means for spraying said pharmaceutical composition to a selected area of said diseased epithelium; and
means for preventing said composition from contacting tissue outside said selected area;
wherein said composition consists of an active component and at least one carrier substance, and wherein said carrier substance is a fluid that becomes highly viscous either upon contact or after a specific time after contact with said epithelium.

24. A device for delivery of components of said pharmaceutical composition of claim 1, comprising at least one chamber for delivery of said components and other substances;
wherein said chambers are separated from each other to avoid mixing of the components and thereby maintain the stability of the components during delivery, and
means for connecting said chambers so that components are mixed prior to treatment.

25. A delivery device according to claim 24, wherein said chambers are manufactured from flexible synthetic material.

26. A delivery device according to claim 24, wherein said chambers are incorporated into a syringe.

27. A delivery device according to claim 24, wherein said device is an improved skin patch containing chambers that are rupturable upon removal of protective layers of said skin patch.

28. An improved skin patch according to claim 27, further comprising:
at least one removable protective outer layer to prevent radiation from penetrating said patch;
means to separate components of said pharmaceutical composition, wherein said means is removable to allow mixing of said components prior to treatment; and
at least one removable protective inner layer to prevent said composition from contacting a treatment area prior to treatment;
wherein said layers are formed from sheets of material that are sealable and further are cut to conform to a size and shape of said treatment area prior to treatment.

29. An improved skin patch according to claim 28 comprising at least one layer, wherein said at least one layer is selected from a group consisting of an adhesive and a plastic film.

30. A pharmaceutical composition according to claim 28, wherein said improved skin patch is transparent to allow irradiation without removing said patch.

31. A pharmaceutical composition for diagnosis or treatment of diseased tissue, including diseased epithelium and affected tissue lying immediately below said epithelium, comprising:
an active component; and
at least one carrier substance;
wherein said carrier substance has sufficient viscosity to remained confined to a specific area.

32. A pharmaceutical composition according to claim 31, wherein said active component is selected from a group consisting of a photosensitizer, photosensitizer precursor, aminolevulinic acid (ALA) and derivatives of ALA.

33. A pharmaceutical composition according to claim 31, wherein said carrier substance is a highly viscous fluid.

34. A pharmaceutical composition according to claim 31, wherein said carrier substance is a gel.

35. A pharmaceutical composition according to claim 31, wherein said carrier substance is a fluid that becomes highly viscous after said substance is applied to said tissue.

36. A pharmaceutical composition according to claim 34, wherein said carrier substance is a thermosetting gel.
